(19) United States
(12) Patent Application Publication
(10) Pub. No.: US 2022/0265336 A1
(43) Pub. Date: Aug. 25, 2022

(54) INSTRUMENT FOR PLASMA SURGERY AND METHOD FOR GENERATING PLASMA

(71) Applicant: Erbe Elektromedizin GmbH, Tuebingen (DE)

(72) Inventors: Tjark Brandt, Tuebingen (DE); Uwe Schnitzler, Tuebingen (DE); Johannes Heym, Tuebingen (DE); Martin Walz, Tuebingen (DE)

(73) Assignee: Erbe Elektromedizin GmbH, Tuebingen (DE)

(21) Appl. No.: 17/628,709

(22) PCT Filed: Jul. 21, 2020
(86) PCT No.: PCT/EP2020/070585

§ 371 Date: Jan. 20, 2022

(30) Foreign Application Priority Data
Jul. 23, 2019 (EP) 19187863.6

(51) Int. Cl. A61B 18/04

(52) U.S. Cl. CPC A61B 18/042; A61B 2018/00589

(57) ABSTRACT

An electrosurgical instrument having an electrode arranged in a gas-carrying lumen and retained in a centred position. The electrode has an electrode body made of a thermally stable material, for example, hard metal, tungsten, steel, stainless steel or similar. The electrode has a coating made of a material with a low melting point, such as silver, silver alloys or another metal with a low melting point. A bonding layer, in particular a gold layer, can be provided between the coating and the electrode body.

The invention refers to an electrosurgical instrument, particularly for plasma treatment of biological tissue, in particular for plasma coagulation, as well as a method for plasma generation.

A plasma coagulation instrument having an electrode that is configured as flat platelet and further a plasma coagulation instrument having a wire-shaped electrode, both arranged in a lumen of a hose-like fluid conductor respectively, are known from DE 100 30 111 A1. Due to the shaping, the electrodes are respectively supported at the inner wall of the lumen such that the tip of the respective electrode is centered and immovably held inside the lumen. The electrical discharge originating from the electrode forms a plasma jet together with gas flowing through the lumen.

Further, a similar instrument is known from WO 2005/046495 A1 having a wire or pin-like electrode that is immovably centrally held in a lumen of a hose-like fluid conductor. A metal platelet extending diametrically through the channel being supported with its longitudinal edges at the inner wall of the lumen, on which the electrode is attached, serves to fixate the electrode. For example, the electrode is formed by a tungsten wire.

Furthermore, an electrosurgical electrode for contact coagulation is known from EP 1 743 588 B1 comprising an electrode body that consists of molybdenum in the core and is plated with a silver alloy. The silver alloy consists of silver having 1.4% to 4% germanium and 1% to 2% indium. With such electrodes impairments occurring on the tissue during tissue cut shall be mitigated.

During operation of the plasma coagulation instruments a remarkable heat creation occurs at the electrode and in its environment. The electrode is typically arranged in a fluid conductor that can be affected by the created heat. Also the electrode itself can be affected. For minimizing or avoiding such impairments, a thermal protection of the fluid conductor has been provided in the past. DE 100 30 111 A1, mentioned above, proposes the configuration of the electrode as flat platelet for improvement of its cooling. Due to the two-dimensional configuration of the discharge section of the electrode, the heating of the probe shall be avoided. On the contrary according to WO 2005/046495 A1, a ceramic tube shall be attached on the distal end of the fluid conductor that keeps the occurring heat development away from the plastic hose of the probe.

All mentioned measures limit miniaturization of the instrument, particularly in the case of a plasma coagulation instrument.

Starting therefrom it is the object of the invention to provide a concept with which the dimensions of plasma coagulation probes can be further reduced.

This object is solved with the electrosurgical instrument according to claim 1 as well as the method according to claim 14:

The instrument comprises a fluid conductor having at least one lumen in which an electrode is arranged, preferably immovably and preferably in a centered manner. The electrode consists of an electrode body that extends starting from its distal end in proximal direction inside the lumen and on which a coating is arranged. The lumen can be connected to a gas source, particularly an argon source, such that the electrode is arranged in a fluid stream. During operation the instrument can emit an axially or also laterally orientated plasma jet at the distal end. The lumen can be realized as channel of a hose or tube. The hose or tube can comprise one or multiple lumen.

The electrode is at least partly arranged inside the lumen and is for this purpose axially displaceable or axially immovable preferably in a centered manner. For example, the electrode can be attached to a holder for this purpose extending up to the limitation surface of the lumen. As an alternative, the electrode itself can extend up to the limitation surface in order to be supported thereon.

In an embodiment of the invention, the distal end of the electrode is located inside the lumen. The plasma jet is thus created within or at the distal end section of the lumen. The end of the electrode does not project out of the lumen and is thus protected from direct tissue contact.

However, the electrode can also be placed such that its distal end projects out of the lumen. Particularly the electrode can support an insulator on its distal end. The insulator consists preferably of an electrically insulating ceramic material.

The insulator can be configured as ball that is supported by the electrode. The insulator can also be configured as hemisphere supported by the electrode. Thereby the insulator can face the lumen with its rounded or flat side. The insulator can also be configured as cylinder rounded at its end(s). Furthermore, the insulator can be configured as circular cone having a rounded or planar base. Thereby the insulator can face the lumen with its base or its conical side. The insulator can also be configured as disc. Preferably the insulator is rotationally symmetrically configured and arranged relative to the wire-shaped electrode.

A laterally orientated opening can remain between the insulator and the distal end of the hose or tube through which gas and/or plasma can exit. The opening can extend along 360° around the electrode. For example, it can be configured as ring-shaped slit or as wide ring-shaped clear region between the insulator and the hose or tube end. In this case the insulator is supported only by the electrode. It is not connected with the tube or hose or with elements arranged therein.

The laterally orientated opening can also be separated in two or multiple partial openings. For example, one, two or multiple webs can connect the insulator with the hose or tube inside which the lumen is configured and the electrode is arranged. The web or webs can be connected with an element arranged inside the lumen or an element arranged outside on the tube or hose, for example a holder. The holder can be monolithically configured of ceramic material with the webs and the insulator.

In embodiments in which the electrode extends out of the lumen, the part of the electrode on which discharge root points of the plasma discharge are created, can be entirely arranged outside of the lumen. For example, the electrode can project 1 mm to 3 mm out of the lumen.

Independent from whether the electrode projects out of the lumen or is arranged entirely therein, it applies:

The coating arranged on the electrode body coats the electrode body, wherein in a distance of multiple millimeters from the distal tip the cross-section of the coating is at least 12% of the cross-section area of the entire electrode. Preferably this applies for a remarkable portion of at least some millimeters extending away from the distal end of the electrode in proximal direction of at least some millimeters, independent from the electrode shape.

According to the invention, the coating is reduced or eliminated at the distal end of the electrode independent from the geometric shape of the electrode, at least after some time of intended use and during intended use. Alternatively, the distal end of the electrode can also be free from the coating or the coating can have a reduced thickness at the distal end or can be interrupted one time or multiple times already prior to the first use. In the context of the present description the distal end is considered as an end section of the electrode that can have a length of 1 mm to 3 mm. The distal end can be configured to be blunt, rounded, tapered or pointed.

The electrode can be configured as platelet having a distal tip or also as pin or needle-shaped electrode (so-called wire electrode). The electrode is arranged by suitable means, e.g. a holder, inside the lumen of the fluid conductor, preferably immovably or also longitudinally movably. It is preferably centered.

The coating of the electrode comprises preferably a melting temperature that is lower, preferably remarkably lower than the melting temperature of the electrode body. The electrode body consists preferably of a material having a melting point above 1000° C. It can consist of steel, stainless steel, particularly chromium and/or nickel comprising steels, molybdenum, tungsten, hard metal or another preferably electrically conductible material. The electrode body consists preferably of a carbon containing metal or a carbon containing metal alloy. The carbon content is preferably larger than 0.02% by weight, preferably at least 0.05% by weight

On the contrary, the coating preferably comprises a low melting temperature of less than 1000° C. Thereby the melting temperature is preferably selected so low that during conditions of use for which the instrument is provided, at least a part of coating material at the distal end or in the proximity of the distal end of the electrode melts. The conditions of use for which the instrument is provided refer to the considered gas flows and electrical powers during the use on the patient. The coating material can be preferably silver or a silver alloy. Particularly a material is preferred as coating material that does not or only to a minor extent react with the gas flowing inside the lumen.

By means of the mentioned measures, an electrode structure can form at the start of the use of the instrument in which the coating of the electrode at the distal end is missing or comprises a structure that deviates from the structure of the remaining coating. In doing so, it can be achieved that the electrical discharge is concentrated on the distal end of the electrode. Thus, also the thermal introduction into the electrode is concentrated on the distal end thereof. In doing so, the electrode absorbs remarkably less heat than known electrodes in which the discharge root point travels or jumps along the electrode.

The electrode can comprise a rough surface at its distal end at least after the initial use at which at least some islands exist that are cleared from the coating material. Preferably the electrode base material comprises carbon that forms accumulations at some locations and is bare at some locations. The carbon can be surrounded by coating material or can be located in areas that are cleared from coating material. Carbon cluster can form discharge root points.

The named effects occur particularly in case of electrode diameters below 0.5 mm and coatings, the thickness of which exceeds a minimum dimension. The minimum dimension is achieved, if at least 10% or better 12% of the electrode cross-section area consists of coating material.

During use the coating can melt particularly at the distal end and can form an entirely or partly liquid area. The coating can slightly retract from the distal end of the electrode body and make the electrode body entirely or partly bare. However, an increased electrode lifetime shows with reduced material removal compared with non-coated electrodes.

It shows that the introduction of thermal energy into the electrode is minimized due to the concentration of the electrical discharge on the outermost distal end of the electrode. The effect is so strong that at least in some embodiments the fluid conductor can be entirely made of plastic and ceramic coating can be avoided also at the distal end. Moreover, it is possible to give up cooling of the electrode by means of cooling bodies or other measures. A thermally and/or electrically insulating material, such as ceramic or plastic, can be used as electrode holder without having to fear damage of the instrument also during longer use.

It also contributes to this behavior, if the electrode has a section at its distal end having a length of at least 2 mm, preferably at least 2.5 mm, for example, the thermal capacity of which is less than 4.5 mJ/K, preferably less than 4.17 mJ/K. The low thermal capacity contributes to the localization and fixation of the discharge at the distal end of the electrode. The electrode reaches its operating temperature quickly at its distal end at which the coating metal is liquid, at least in sections. It can retract from the hot electrode tip. The discharge root point is fixed on the hot electrode tip. It does not travel in proximal direction and particularly does not cross the forming ring-shaped barrier of melted coating metal (e.g. silver).

A strong axial temperature gradient having a strong decrease between high temperature at the distal end and low temperature beyond an approximately ring-shaped area, in which liquid coating material is present during use, is obtained on the electrode.

Also a concept for forming the electrode with anchoring of the discharge root point or the discharge root points at the distal end of the electrode is part of the invention. The discharge root points are points with increased electron emission compared with the environment. They can be recognized as locations from which visible, strongly shining strands originate inside the plasma.

The desired configuration of the electrode can also occur by forming of the electrode during initial use. A still unused electrode can have an extensively precisely defined geometric shape, particularly at its distal end. The coating can extend with extensively constant thickness up to the outermost distal end of the electrode. No later than during the initial use one or multiple areas of the distal end of the electrode can be bare or can have a reduced thickness of the coating. Thus, the electrode surface at the distal end distinguishes from the electrode surface in sections that are located farther proximally. The differences can be of material or structural nature. Particularly, the distal end of the electrode can comprise carbon particles on the surface. The differences of the surface of the distal end compared with the surface of the other electrode result in fixing the discharge root point on the outermost distal end of the electrode and thus in minimizing the thermal introduction into the electrode and the surrounding wall of the fluid conductor.

Further details of preferred embodiments of the invention are derived from the drawings and the following description. The drawings show:

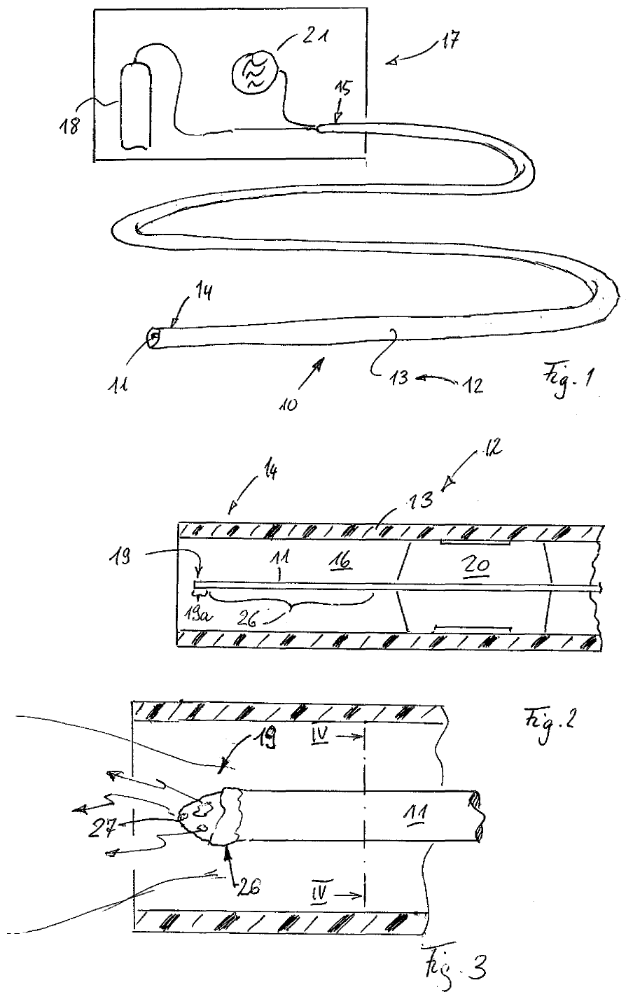

FIG. 1 an instrument and the apparatus provided for supply in a schematic illustration,

FIG. 2 a distal end section of the inventive instrument in enlarged longitudinal section basic illustration,

FIG. 3 a distal end section of the instrument according to FIG. 2 during operation,

FIG. 4 a cross-section through the electrode according to FIG. 3 cut along the line IV-IV,

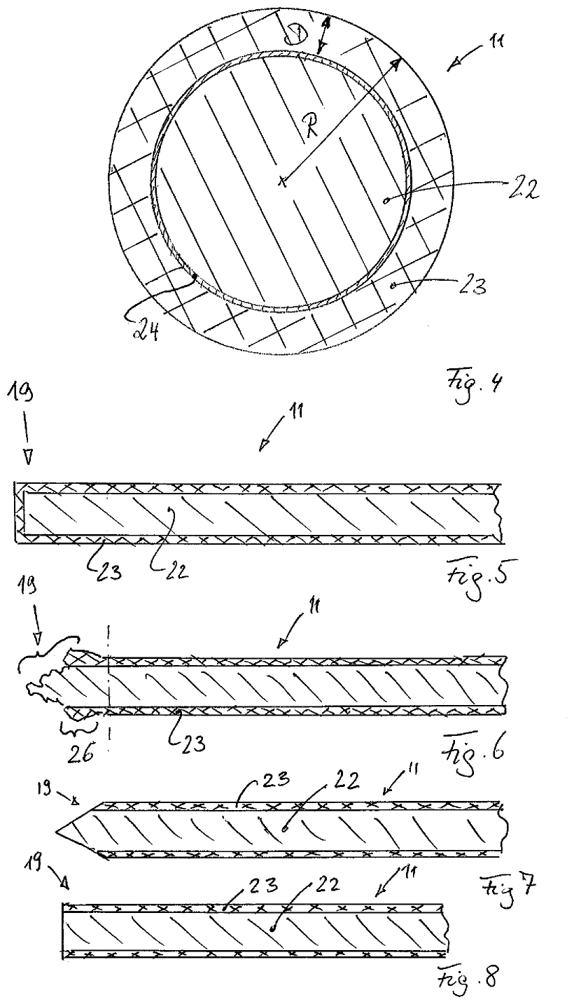

FIG. 5 an electrode in longitudinal section illustration of its distal end section prior to its first use,

FIG. 6 the electrode according to FIG. 5 in formed condition,

FIGS. 7 and 8 additional embodiments of an inventive electrode prior to the first use in longitudinal section.

FIG. 1 illustrates an instrument 10 that is configured as endoscope probe. It serves for plasma coagulation, particularly argon plasma coagulation, i.e. for treatment of human or animal tissue without direct physical contact between its electrode 11 and the respective biological tissue. The instrument 10 is configured as flexible probe. The principals explained in the following can, however, also be realized in a rigid instrument suitable for the laparoscopic use or in an instrument suitable for the open surgical use.

The instrument 10 comprises a fluid conductor 12, e.g. in form of a flexible hose 13, but extends from a distal end 14 up to its proximal end 15. A lumen 16 extends through the length of hose 13 that is particularly apparent from figure 2. During use a gas, typically an inert gas such as argon, flows through this lumen. For this, instrument 10 is connected to an apparatus 17 that comprises a gas source 18 or provides a connection to such a gas source. During use gas flows from proximal end 15 to the distal end 14 of hose 13 and thus of lumen 16 and therefrom out of the open end of hose 13.

The electrode 11 is arranged in the lumen 16, the distal end 19 of which preferably does not project from the fluid conductor 12, but is rather still positioned inside lumen 16. However, it can also extend with a section out of the fluid conductor 12 and/or lumen 16 in some embodiments. Electrode 11 is preferably a pin or needle electrode that can be configured, for example, by a round or profile wire or also by a small tube or cannula. The electrode 11 can also be the distal end of the wire extending through the lumen 16. The electrode 11 can comprise a substantially constant cross-section. It can be held inside lumen 16 preferably in a centered manner and immovably or axially movable independent from its specific shape. The holder 20 provided for this purpose supports electrode 11 and is supported on the inside of fluid conductor 12 or hose 13.

The electrode 11 is electrically connected with a generator 21, e.g. an RF generator, that supplies a radio frequency electrical voltage to the electrode 11. A respective connection conductor can extend from electrode 11 through the entire lumen 16 up to the proximal end 15 at which an electrical connector provides the contact with generator 21.

Generator 21 is preferably configured to supply a voltage that is sufficiently high in order to create an electrical discharge at the tip of electrode 11 and in doing so, to ionize the gas stream flowing along electrode 11 at least partly. A plasma jet for treatment of biological tissue is created.

A decisive particularity of the present instrument 10 is the characteristic of electrode 11. It is, for example, configured as slim cylinder having a flat, round, cone-shaped or tapered tip or entirely as slim cone. It comprises preferably a diameter of less than 0.5 mm, preferably at most 0.3 mm at least in the proximity of its distal end 19. Thus, its radius R, apparent from FIG. 4, is smaller than 0.25 mm, preferably smaller than 0.15 mm. Radii smaller than 0.1 mm are possible. Electrode 11 can, however, also comprise a prismatic shape, e.g. in that it is configured as profile wire. In addition, it can be configured as flat platelet having a tip orientated in distal direction.

FIG. 4 illustrates a cross-section of electrode 11 in an axial distance of some millimeters toward the distal end 19 of the electrode 11. The distance is so long that the structure of electrode 11 remains unchanged during use. As apparent, electrode 11 comprises an electrode body 22 that is provided with a coating 23. The electrode body 22 and the coating 23 consist of different materials. Preferably the melting temperature TK of the electrode body 22 is above 1000° C. Also the electrode body 22 can consist of another thermal-resistant electrically conductive or also—at least in cold condition—of an electrically non-conductive material, such as ceramic, for example.

Electrode body 22 can consist of a high-melting metal such as, for example, steel, stainless steel, hard metal, molybdenum, tungsten or the like. Particularly alloys are suitable as material for the electrode body 22 that contain ion and/or chromium and/or nickel. Moreover, carbon and/or manganese and/or phosphor and/or sulphur and/or silicon and/or nickel and/or nitrogen and/or molybdenum can be present as additional alloy components. A stainless steel preferred as base material has the following composition:

| | Fe | C | Cr | Mn | P | S | Si | Ni | N | Mo |
|---|---|---|---|---|---|---|---|---|---|---|
| min | | 0.05 | 16.0 | | | | | 6.0 | | |
| max | 47.605 | 0.15 | 19.0 | 2.0 | 0.045 | 0.15 | 2.0 | 9.5 | 0.11 | 0.8 |

On the contrary, coating 23 preferably consists at least partly of an electrically well conductible low-melting material having a melting point preferably lower than 1000° C., the coating 23 can consist of silver or silver alloys, for example. The thickness D of coating 23 is preferably at least so thick that the portion of the area of the cross-hatched cross-section area of coating 23 in FIG. 4 of the total cross-section area of electrode 11 is higher than 10%, preferably higher than 12%. The total cross-section area is the cross-section area having the area of a circle with radius R. This corresponds to the sum of the area of the cross-section of the electrode body 22 (obliquely hatched in the figure) and the area of the cross-section of the coating 23 (cross-hatched in FIG. 4) in FIG. 4. The explained correlation between the cross-section area of the coating 23 and the total cross-section area of electrode 11 applies independent from its specific cross-section shape. Thus, electrode 11 can comprise a hollow cylindrical or a polygonally limited cross-section.

An intermediate layer 24 can be provided between the coating 23 and the electrode body 22. It can consist of a metal, preferably a noble metal, a noble metal alloy or an inert metal, e.g. nickel, a nickel alloy, gold or a gold alloy. The melting temperature T_{z} of the material of intermediate layer 24 is preferably between the melting temperatures T_{K} and T_{B} of the materials of the electrode body 22 and the coating 23 (T_{K}>T_{Z}>T_{B}). The intermediate layer 24 can act as adhesive and concurrently support the retraction of the melting coating 23 from electrode body 22 at the distal end 19.

Advantageous thermal conditions result when complying with the indicated parameters, i.e. diameter of the electrode 11 smaller than 0.3 mm and portion of the cross-section area of the coating 23 larger than 10%, preferably larger than 12% of a total cross-section area of electrode 11. Instruments 11 having filigree outer dimensions can be configured in this manner. The outer diameter of the fluid conductor 12 can be 1 mm or less if required.

The obtained miniaturization possibility is based on the low thermal development and thermal radiation at and from electrode 11. This is achieved by the combination of at least some of the measures described above. In doing so, it is particularly achieved that the electrical discharge during operation is concentrated on the distal end 19 of electrode 11. It comprises a section 25 adjoining the distal end 19 that has a length of preferably several millimeters. Inside it the conditions with regard to the cross-section areas of the electrode body 22 and the coating 23 described in connection with FIG. 4 apply. Preferably section 25 ends proximally ahead of or at holder 20. The coating can, however, extend and can be continued beyond holder 20 in proximal direction. In distal direction section 25 ends at the distal end 19 of electrode 11. The distal end 19 starts at an area 26 apparent from FIG. 3 that is located so close to the forming discharge root points 27 that material of the coating 23 in this area 26 is present or can be present in liquid condition during operation.

At the distal end 19 at least parts of electrode body 22 are bare during operation. The bare area forms the distal end 19. Starting from the face side end of electrode 11 up to approximately 2 mm to 2.5 mm in proximal direction, a section 19a is formed, the thermal capacity of which is preferably less than 4.5 mJ/K and further preferably less than 4.17 mJ/K. The section 19a can be formed by the distal end 19. The low thermal capacity of section 19a allows local melting of coating 23. Also the continuous electron emission of end 19 is guaranteed immediately after ignition of a plasma also with low RF power. This supports the concentration of the plasma discharge on the distal end of the electrode and thus mitigates thermal introduction therein.

The instrument 10 described so far is used as follows and its electrode 11 is operated as follows:

In operation first lumen 16 is supplied with gas, such that a gas flow results in distal direction. For example argon can serve as gas that longitudinally flows along electrode 11. Electrode 11 is electrically connected with generator 21. The voltage applied to the distal end 19 results in a spark ignition to a counter electrode located in the proximity that can be biological tissue, for example.

Directly before or after start of this process, electrode 11 has the initial shape illustrated in FIG. 5 that is geometrically defined. For example, electrode body 22 is cylindrical, while coating 23 essentially has a constant thickness everywhere. Coating 23 extends starting from the distal end 19 some millimeters or centimeters in proximal direction and can end then or can be extended. Coating 23 can extend over the face of electrode body 22 or can leave it bare, as apparent from FIG. 8, for example. Coating 23 can also already be removed from an end section, e.g. from the distal end 19, during production of electrode as shown in FIG. 7. For this, distal end 19 of electrode 11 can be conically pointed, configured as truncated cone or also as wedge. For example, electrode 11 can be manufactured by cutting a sufficiently long part from an endlessly supplied coated wire. As necessary, distal end 19 can be processed subsequently in a material removal manner in order to remove coating 23 at the end 19 completely or partly.

With the first initial use, first distal end 19 of electrode 11 is heated such that it is reshaped for the further operation. An area 26 is formed on electrode 11 in which material of coating 23 is at least partly melted, as illustrated in FIG. 6. In this area 26 coating can be thicker than in the remaining region of electrode 11. On the contrary, coating can have a lesser thickness, can be interrupted or can be completely missing at the outermost end 19.

The procedures described above in connection with the first initial use can also be carried out in the context of the manufacturing of instrument 10. For this, the manufacturer can operate instrument 10 under controlled conditions for a short period. The manufacturer can thereby define the gas type and gas flow as well as voltage and current, just as for the operation on the patient. It can, however, also select different gas types, gas flows or operating voltages or currents.

During operation distal end 19 of electrode 11 is heated and able to emit electrons, while electrode 11 reaches remarkably lower temperatures in the area 26 and particularly farther proximally in section 25 and remains relatively cool there. At the distal end 19 discharge root points 27 are fixed (FIG. 3) without travelling in proximal direction. Electrode 11 thus radiates only low heat and does not substantially contribute to heating the fluid conductor 12.

The instrument 10 according to the invention comprises an electrode 11, which is arranged in a gas-conducting lumen 16 and held in a centered position. Electrode 11 comprises an electrode body 22 made of a thermally stable material, e.g. hard metal, tungsten, steel, stainless steel or similar. The electrode 11 is provided with a coating 23 made of a material with a low melting point, such as silver, silver alloys or another metal having a low melting point. An adhesive layer 24, particularly a gold layer, can be provided between the coating 23 and the electrode body 22. It can facilitate the retraction of coating 23 during initial use and thus the formation of electrode 11 desired for operation (e.g. according to FIG. 6).

### Reference Signs

10 instrument

11 electrode

12 fluid conductor

13 hose

14 distal end of fluid conductor 12/hose 13

15 proximal end of fluid conductor 12/hose 13

16 lumen

17 apparatus

18 gas source

19 distal end of electrode 11

19a section of electrode 11 (2 to 2.5 mm length)

20 holder

21 generator

22 electrode body

23 coating

24 intermediate layer

25 section

26 area in which the coating material can be liquid

27 discharge root points

1. An electrosurgical instrument for plasma coagulation, the electrosurgical instrument comprising:
a fluid conductor that comprises at least one lumen; and
an electrode having a coating and being arranged inside the fluid conductor at least in sections, the electrode comprising an electrode body extending from its distal end in a proximal direction into the fluid conductor.

2. The electrosurgical instrument according to claim 1, wherein the coating comprises a melting temperature and the electrode body comprises a melting temperature and the melting temperature of the coating is lower than the melting temperature of the electrode body.

3. The electrosurgical instrument according to claim 1, wherein the electrode body comprises a material having a melting temperature over 1000° C.

4. The electrosurgical instrument according to claim 1, wherein the coating comprises a material having a melting temperature below 1000° C.

5. The electrosurgical instrument according to claim 1, wherein the coating comprises an inert metal.

6. The electrosurgical instrument according to claim 1, wherein the coating has a cross-section with a coating area and the electrode has a cross-section with an electrode area, wherein the coating area is at least 12% of the electrode area and wherein the coating extends along a section of electrode of multiple millimeters.

7. The electrosurgical instrument according to claim 1, wherein electrode further comprises a section at its distal end of at least 2.5 mm length, the thermal capacity of which is lower than 4.5 mJ/K.

8. The electrosurgical instrument according to claim 1, wherein electrode further comprises a distal end held inside the lumen.

9. The electrosurgical instrument according to claim 1, further comprising a holder for arrangement of the electrode inside the lumen and on which the electrode is held, the holder arranged to center the electrode inside the lumen.

10. The electrosurgical instrument according to claim 9, wherein the holder comprises a poor thermally conductive material.

11. The electrosurgical instrument according to claim 9, wherein the holder comprises an electrically insulating material.

12. The electrosurgical instrument according to claim 1, wherein during operation the coating comprises a melted section in the proximity of the distal end.

13. The electrosurgical instrument according to claim 1, wherein during operation the electrode comprises a section at its distal end that is at least partly bare from material of the coating.

14. A method for generation of a plasma, the method comprising:
providing an instrument according to, claim 1; generating a gas flow inside the lumen of the fluid conductor;
applying an electrical voltage to the electrode for causing an electrical discharge at the distal end of the electrode to heat the distal end of the electrode at least up to locally reaching or exceeding the melting temperature of the coating; and
further operating the instrument.

15. The method according to claim 14, further comprising:
redistributing material of the coating away from the distal end of the electrode while at least partly clearing the electrode body.

16. The electrosurgical instrument according to claim 7, wherein the electrode further comprises a section at its distal end of at least 2.5 mm length, the thermal capacity of which is lower than 4.17 mJ/K.
